(19) United States
(12) Patent Application Publication
(10) Pub. No.: US 2024/0341873 A1
(43) Pub. Date: Oct. 17, 2024

(54) STERILE DISPOSABLE INTERFACES FOR PERCUTANEOUS INSTRUMENTS

(71) Applicant: c/o Mendaera, Inc, San Carlos, CA (US)

(72) Inventors: Jason Tomas Wilson, San Carlos, CA (US); Erica Ding Chin, San Carlos, CA (US); Trent Michael Callan, San Carlos, CA (US); Colin Allen Wilson, San Carlos, CA (US); Ben Dickerson, San Carlos, CA (US); Mary Margaret Scheunert, San Carlos, CA (US); Alex Hassan, San Carlos, CA (US); Polly Ma, San Carlos, CA (US); Joshua DeFonzo, San Carlos, CA (US)

(21) Appl. No.: 18/637,946

(22) Filed: Apr. 17, 2024

Related U.S. Application Data
(60) Provisional application No. 63/459,921, filed on Apr. 17, 2023.

(51) Int. Cl. A61B 34/30; A61B 90/50

(52) U.S. Cl. CPC A61B 34/30; A61B 90/50; A61B 2034/301

(57) ABSTRACT

Robotic intervention systems may be used to perform medical procedures on patients using percutaneous instruments. Percutaneous interventional techniques are typically short in duration, requiring quick set-up and tear down to ensure minimal impact to medical workflow or process while maintaining a sterile field. A sterile disposable interface provides an inexpensive sterile barrier between the interventional site and the robotic system.

### CROSS REFERENCE TO RELATED APPLICATIONS

This application claims priority to U.S. Provisional Application No. 63/459,921, filed Apr. 17, 2023 entitled “STERILE DISPOSABLE INTERFACES FOR PERCUTANEOUS INSTRUMENTS,” which is incorporated by reference herein in its entirety.

### TECHNICAL FIELD

The present invention relates to the field of medical imaging systems.

### BACKGROUND

Medical interventional robotic systems often require establishing a sterile barrier between the interventional site and the capital robotic system. In particular, percutaneous interventional techniques are typically short in duration, requiring quick set-up and tear down to ensure minimal impact to medical workflow or process while maintaining a sterile field. As a result, requiring reprocessing or sterilization of components to establish a sterile field is not tenable, and instead it is more appropriate to use inexpensive sterile disposables. This paradigm allows for quick set up and tear down of sterile equipment through a sterilized disposable pack.

Handheld percutaneous interventional systems are best designed when one hand is dedicated to stabilizing the imager and robot, while the other is dedicated to manipulating the system and instruments to choose an insertion trajectory and perform the intervention. A natural consequence is the desirability to use a single sterile hand to interact with sterile interface to load instruments, manipulate the robot, insert the instrument, and release the system from the needle (optionally while inserted). Further, there may be a need to include other features that necessarily interact with the instrument to calibrate the location of the instrument and measure instrument tip depth relative to the instrument guide or imager.

These requirements conflict with the desire to have the sterile barrier be inexpensive and simple/quick to use. The disclosed invention satisfies the need for a rich user interface as well as supporting sterility and disposability needed to practically perform procedures of a percutaneous nature.

### SUMMARY

Embodiments disclosed herein describe a sterile component of a handheld percutaneous interventional system. In one embodiment, the interventional system comprises an imaging device (e.g., hand held ultrasound probe) which may be coupled to a robotic arm. The robotic arm comprises an instrument guide, which points an instrument (e.g., needle like medical instrument) at a desired target identified on an image generated by the imaging device (e.g., ultrasound image of human anatomy). A sterile instrument interface attaches the instrument (e.g., standard needle-like instruments) to the robotic arm, while providing other capabilities that enable user interaction and robotic manipulation.

In some embodiments, the sterile instrument may comprise a drape to create a sterile barrier around the robot and ultrasound probe. In some embodiments, the sterile instrument may comprise a drape to create a sterile barrier around the robotic arm.

Various embodiments and examples of such robotic systems are described in U.S. application Ser. No. 17/860,970, filed Jul. 8, 2022, which claims priority to U.S. Provisional Patent Application No. 63/219,662 filed Jul. 8, 2021, entitled “REAL TIME IMAGE GUIDED PORTABLE ROBOTIC INTERVENTION SYSTEM.” However, the innovations described herein may operate with other robotic systems as well.

### BRIEF DESCRIPTION OF THE DRAWINGS

FIG. 1 is an example of a sterile drape, with a robotic arm, and instrument interface;

FIG. 2 is an example of the instrument interface;

FIGS. 3-4, are examples of a top button;

FIG. 5 is an example of a top lock;

FIGS. 6-7 are examples of a carriage and a carriage brake;

FIG. 8 is an example of a rotation lock;

FIGS. 9-11 are examples of an instrument adapter;

FIG. 12 is an example of an insertion rail;

FIG. 13 is an example of a guide constraining needle motion;

FIG. 14 is an example of a guide lateral release;

FIG. 15 is an example of a homing switch;

FIG. 16 is an example of a lower button;

FIG. 17 is an example of a sterile adapter;

FIGS. 18-22 are examples of an instrument interface latch;

FIGS. 23-24 are examples of drape packages;

FIGS. 25-28 are example of workflow for rapid installation of a full drape package; and

FIGS. 29-30 are examples of the arm only drape.

### DETAILED DESCRIPTION OF THE DRAWINGS

The following description is provided to enable any person skilled in the art to make and use the invention, and is provided in the context of a particular application and its requirements. Various modifications to the embodiments may be possible to those skilled in the art, and the generic principles defined herein may be applied to these and other embodiments and applications without departing from the spirit and scope of the invention. One skilled in the relevant art will recognize that embodiments of the inventive concepts disclosed herein can be practiced without one or more of the specific details, or in combination with other components, etc. In other instances, well-known implementations or operations are not shown or described in detail to avoid obscuring aspects of various embodiments of the inventive concepts disclosed herein. Thus, the present invention is not intended to be limited to the embodiments shown, but is to be accorded the widest scope consistent with the principles, features and teachings disclosed herein. In the following description, several specific details are presented to provide a thorough understanding of embodiments of the inventive concepts disclosed herein.

Embodiments disclosed herein describe a sterile boundary (or sterile barrier), comprising one or more highly compliant (e.g., drape-like) sections and one or more rigid (e.g., plastic-or rubber-like) sections to limit transmission of particles from robotic system. In some embodiments, sterile boundary may be used to maintain a sterile barrier between the health care provider (HCP), patients, and all non-sterile equipment (e.g. ultrasound probe) during procedures.

The sterile boundary may couple to key features of the robotic device and may include transparent elements to permit visualization of a screen/user interface and to allow touch signal to pass through it to enable interaction with a touchscreen. The sterile barrier may comprise integrated coupling gel. In some embodiments, the sterile barrier may also cover an instrument guide (e.g., a physical needle guide), or interface with a sterile guide tightly to create a sterile barrier.

In some embodiments, a sterile boundary may be used during every medical procedure. In some embodiments, the sterile boundary (or portions) may be disposable.

The specific interface between the robotic system and the sterile instrument (e.g., needle) needs to be sterile, and rigidly in contact with the modular robotic arm (so that it can be steered by the robotic arm). In some embodiments, the sterile barrier may be coupled to the instrument guide (e.g., as one of the rigid sections). In some embodiments, the instrument guide may be attach-able to one of the rigid elements of sterile boundary.

In some embodiments, the rigid elements of the sterile barrier may attach to key features of the robotic arm, such as the Imaging Device head (e.g., ultrasound probe head), the robotic manipulator (e.g., needle targeting arm), instrument guide (e.g., a needle guide), a screen and/or user interface, or buttons.

In some embodiments, the sterile barrier and robotic device may have complementary features to support easy attachment and/or removal of the sterile barrier, such as insets into the housing of the robotic device and corresponding rigid features on the drape, snap fasteners on the robotic device and drape respectively, adhesives, elastic or magnets. These interface features may be related to the rigid features described above, or be part of the compliant portion of the drape.

In some embodiments, the sterile barrier comprises a rigid element that would act as an interface between the instrument guide and the instrument being inserted (e.g., a needle).

In some embodiments, the rigid features of the barrier may include one or more highly transparent elements (glass, plastic, polycarbonate, etc.) to support visualization of a screen/user interface that is included in the robotic device, and to allow capacitive or resistive touch signal to pass through it so that a user can interact with user interfaces included in the robotic device. In some embodiments, the highly transparent elements may be used for other aspects, including sensors, lights, cameras, or other components that may require transparency. In some embodiments, the highly transparent elements may be compliant components of the drape itself.

In some embodiments, the sterile barrier may include integrated cartridges, pockets, or free-floating acoustic coupling gel, which additionally may be enabled to come in contact with the robotic device via removal of adhesive coverings, pull tabs, etc. Integration of a gel cartridge, pocket, or even free-floating gel in the housing of the drape may enhance workflow dramatically. In some embodiments, the pockets may be covered (e.g., using pull-tabs or adhesive layers), such that the user remove the cover adhesive layer and expose the gel, thereby allowing contact between the probe and the gel, and thus ensuring acoustic coupling.

In some embodiments, the sterile barrier may include an integrated instrument guide. For example, the sterile barrier with instrument guide may be used with embodiments of a robotic arm without an instrument guide. In some embodiments, the sterile barrier may comprise unique features on the instrument guide and/or robotic arm to ensure the instrument guide is properly secured to the robotic arm and that the system has an accurate understanding of the location of the instrument (e.g., needle), and the trajectory it would follow when inserted into an object (e.g., anatomy). In some embodiments, sterile barrier may include an integrated instrument guide and an integrated instrument. For example, a needle with syringe preloaded may be incorporated into a sterile barrier with integrated instrument guide as one piece.

During a procedure, the user may hold the imaging device (e.g., ultrasound probe) in one hand, and manipulate the modular robotic arm, instrument and/or instrument interface with the other. Therefore, an instrument interface should provide a rich set of features including: Interfacing with the robotic arm, interfacing with (e.g. holding) the medical instrument, integrating with a drape to provide a sterile field, exposing buttons and other touch points to the user to manipulate the robot, and measuring various states of the instrument to provide robotic functionality (e.g. instrument tip measurement).

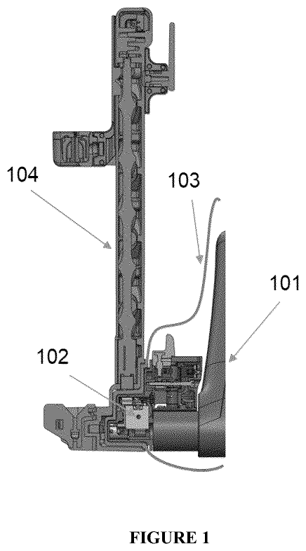

FIG. 1 is an example of a modular robotic arm, sterile drape, and instrument interface. A modular robotic arm 101 may comprise one or more sensing electronics, for example, a printed circuit board (PCB) 102. A sterile drape 103 may cover the robotic arm 101 and couple to an instrument interface, for example, a sterile disposable piece 104.

In some embodiments, the instrument interface may comprise a passive mechanical apparatus that is an assembly of simple mechanical components. In some embodiments, each component may be made of inexpensive plastic or stamped metal components that are compatible with sterilization processes. In some embodiments, some subsystems may require interactions with electronics (e.g., sensor PCB) to measure the state or position of a mechanical component to facilitate functionality like button presses or instrument depth measurement. In these cases, the electronics are housed inside the capital non-sterile structure of the robot and may transmit information across the sterile barrier via mechanical manipulation or electric/magnetic field manipulation.

The sterile field may be created by segregating the modular robotic arm 101 behind a sterile drape 103 that is in between the sterile disposable piece 104 and modular robotic arm 101 and sensor PCB 104 The sensor PCB 104 may interact with the sterile disposable piece 104 to provide a relative location of a percutaneous instrument to the robot arm 101 across the sterile drape 103.

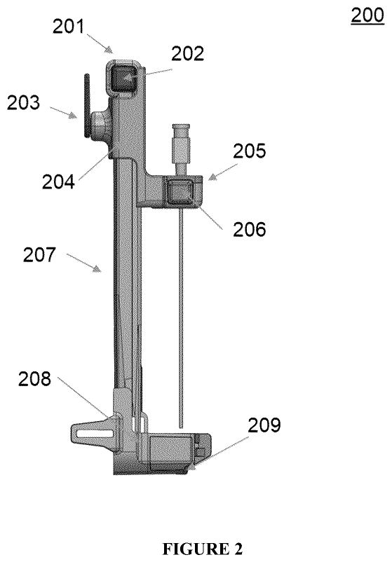

FIG. 2 is an example of the instrument interface. The instrument interface 200 may include multiple disposable components. For example, the disposable components may include a top button (or pitch button) 201, a top lock 202, a carriage brake 203, a carriage 204, a rotation lock 205, an instrument adapter 206, an insertion rail 207, an instrument guide 208, and a needle tip homing 209 (described in details below).

The top button (or pitch button) 201 may facilitate adjusting the robot arm through “backdriving” by a button, which may be located in various locations, including on the back of the insertion rail. The top button 201 may inform the robotic arm that the user wants to manually adjust the desired trajectory of the instrument by directly manipulating the arm. In some embodiments, the location of top button 201 at the top of the rail may be convenient for the user to reach when holding the back of the instrument.

The top lock 202 may lock the carriage 204 of the insertion mechanism at the top of the rail. This may be useful during instrument installation, where it is convenient to have the carriage 204 of the insertion mechanism held at the top of the rail without needing any additional user interactions. In some embodiments, the top lock 202 may comprise a magnet, or detent, or high friction feature at the top of the rail may hold the carriage in place for instrument installation.

The carriage brake 203 (or insertion brake) may lock the carriage 204 to the rail to maintain instrument position relative to the robotic arm. This may be useful when the user may need to perform additional instrument manipulations while the robot holds the instrument at some depth during a procedure.

The carriage 204 may constrain the back of the instrument to stay aligned with the instrument guide while allowing the instrument to travel along the rail. This secures the instrument to the instrument interface, and is a structural support which is particularly important for small diameter needles. The carriage 204 may also interact with the rails internal mechanisms to measure change in needle insertion depth.

The rotation lock 205 may provide resistance to instrument (e.g., needle) rotation, which may be helpful when the user needs to remove or install an accessory (e.g. a syringe from a needle) by a rotating interaction, using one hand.

The instrument adapter 206 is a component which may facilitate one handed quick installation of the instrument on the carriage 204 of the rail. In some embodiments, the instrument adapter 206 may be pre-installed on the needle of the instrument. One end of the instrument adapter may grip the instrument shaft, while the other side has quick attach/release features for a quick and controlled installation.

The insertion rail 207 may constrain the carriage 204 to move along an axis parallel to the instrument guide. The insertion rail 207 may also provide structure to hold the back of the instrument (e.g., needle). In some embodiments, the insertion rail 207 may house a large pitch helical screw that is spun by the carriage 204 as the instrument (e.g., needle) moves along the rail. This rotation may be measured by electronics on the capital side of the robot to directly measure motion of the instrument.

The instrument guide 208 may provide a precision constraint on the motion of the instrument along the axis of the instrument (e.g., needle). To target anatomy, the robotic arm may align the instrument guide with the anatomy of interest.

The instrument depth measurement may be used to indicate to the user the location of the instrument tip on the ultrasound image as it is inserted towards the anatomy of interest. However, the length of the instrument may be unknown to the robotic arm. A homing switch, or needle tip homing 209, may measure the location of the instrument tip as it passes through the instrument guide to calibrate the depth measurement to a tip location for display on a user interface.

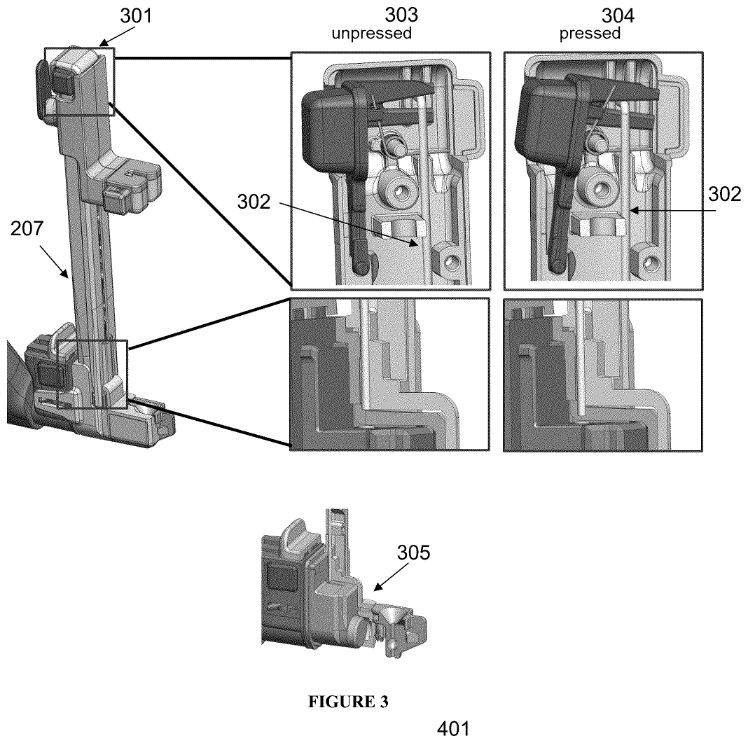

FIG. 3 is an example of a top button. A top button (or pitch button) 301 may facilitate adjusting the robot arm through “backdriving” by a button, which may be located in various locations, including on the back of the insertion rail 207. The top button 301 may inform the robotic arm that the user wants to manually adjust the desired trajectory of the instrument by directly manipulating the arm. The robot arm may respond to the button press by deenergizing the robot arm actuators to facilitate backdriving. In some embodiments, the arm may allow constrained backdriving using impedance or admittance control. In some embodiments, the location of top button 301 at the top of the rail is convenient for the user to reach when holding the back of the instrument. A common interaction may be to keep the needle pointed at the center line of the probe, and constrain adjustment to pitch only, resulting in this feature casually being referred to as a “pitch button.” However, the degrees of freedom that the button enables for backdriving is computer programmable and may be arbitrary.

The top button 301 may communicate button presses to electronics—a button or other electromechanical sensor—inside of the robotic arm across the sterile boundary by way of a pogo stick (or actuation member) 302. A pogo stick (or actuation member) 302 may comprise a long thin push rod that goes down the length of the rail and is aligned with the appropriate electronics. A capital-side pitch button 305 may be located below the pogo stick 302, which may receive the button presses or actions of the top button 301.

In some embodiments, the pogo stick 302 may be translated in a longitudinal direction by a variety of motions-including a top down motion 304, where the top button 301 moves parallel, or mostly parallel to the motion of the pogo stick 302. This motion of the top button 301 may be created through compression or torsion springs, or flexures, or the like.

FIG. 3 further illustrates the different location of the pogo stick 302 when the top button 301 has yet to be pressed 303 versus when the top button 301 is pressed 304.

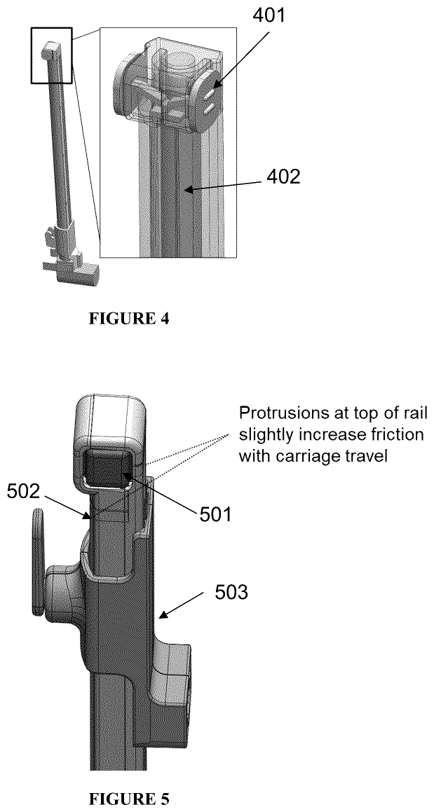

FIG. 4 is another example of the top button. In this example, the rod 402 may be held in tension, such that when the top button 401 is pressed, the rod 402 is pulled off a corresponding button inside of the robot. Other embodiments may include embedding inexpensive electronics near the touch point and creating an electrical connection across the sterile barrier.

In some embodiments, top button 401 may also be side actuated, using cam surfaces or flexures. The advantage of activating the top button in a pinching motion, is that this motion is less prone to accidentally trigger the button when removing an instrument. There's also less chance of the user accidentally translating the rail into the patient.

When installing the instrument to the carriage, it may be important for the carriage to be fully retracted and held still. This allows easy access to the attachment features on the carriage, and keeps the tip of the needle above the needle guide. Similarly, when removing the needle, it may be important for the carriage to stay stationary and at the top of the rail, as opposed to it falling down to the bottom. Further, when installing and removing the needle, the user only has one hand free requiring this mechanism to be automatically activated.

FIG. 5 is an example of a top lock. The top lock 501 may catch the carriage 503 when it is brought in contact with the top surface of the rail, which may be accomplished in a number of ways. In some embodiments, magnets, detents, or high friction features (for example protrusions 502 at the top of the rail may slightly increase friction) are mechanisms which may be used in this application. The user may simply push the carriage to meaningfully interact with the magnetic field or detent mechanism to have the carriage held at the top of the rail. Similarly, the carriage may be moved towards the needle guide to insert the needle by overcoming the magnetic forces or detent spring with the same inserting motion/force applied by the user. Other methods to hold the carriage at the top of the rail include other more involved mechanical interactions including latches, bayonets, pins and the like.

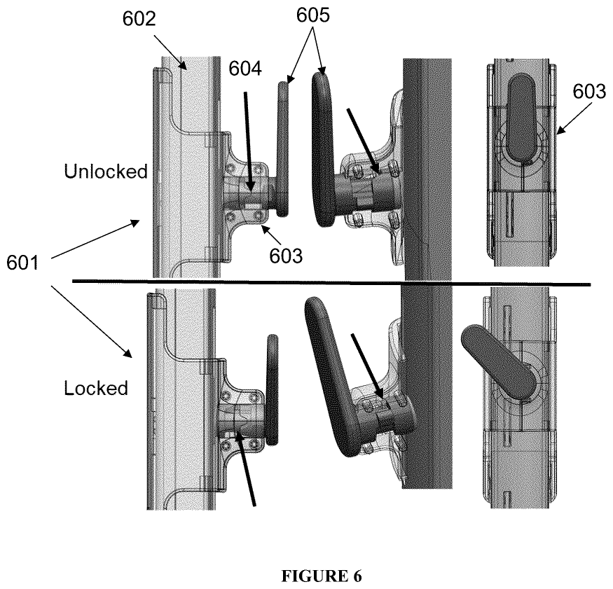

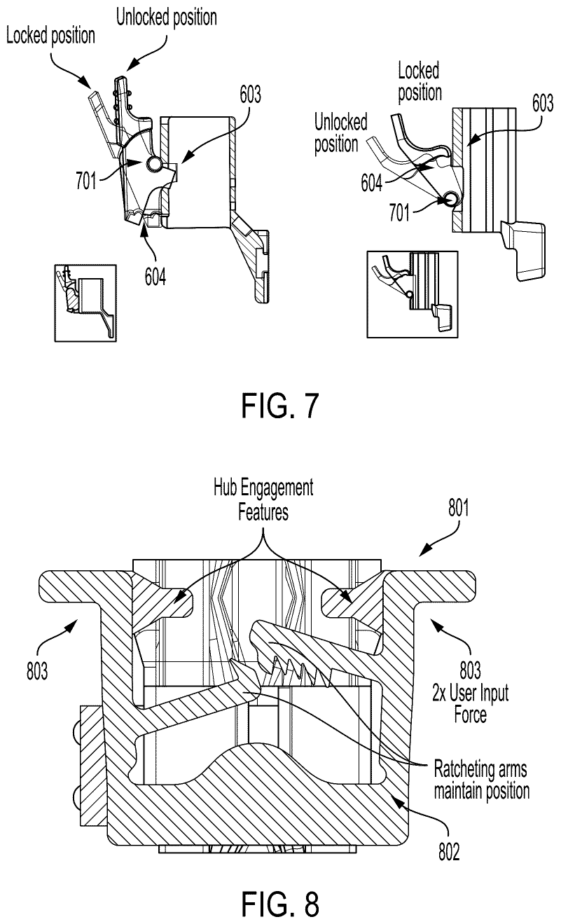

FIGS. 6 and 7 are examples of the carriage and carriage brake. The carriage 601 may constrain the back of the instrument to stay aligned with the instrument guide while allowing the instrument to travel along the rail 602. The carriage 601 secures the instrument to the instrument interface, and is a structural support which is particularly important for small diameter needles.

In some embodiments, the carriage 601 may be designed to have low sliding friction along the rail 602 to facilitate unencumbered motion of the instrument (e.g., needle) during insertion and retraction. However, once the instrument is placed, it is often important to lock the instrument at a certain depth. This may be achieved by locking the carriage 601 to the rail using the carriage brake 603. This may facilitate the removal or installation of an instrument accessory (e.g. syringe). This feature is also useful when the user wants to install or remove the instrument with the carriage 601 located away from the top lock (e.g. short needle cases).

As illustrated in FIGS. 6 and 7, a mechanical friction lock (an example for the carriage brake 603) is shown on the back of the carriage 601. When actuated, the lock interferes with the back of the rail 602 and increases friction to lock the carriage 601 in place. This may be a smooth interaction, or combined with a detent 604 to give the user tactile feedback indicating sufficient locking pressure has been applied. The detents 604 may also be oriented on the same or opposite sides of the touchpoint. As illustrated in FIG. 6, the detents 604 are located on the same side of the lever 605 as the brake 603 side of the paddle relative to the pivot. FIG. 6 shows the carriage brake 603 in both the unlocked and locked positions to show where the lever 605 would be positions and how the detents 604 move.

FIG. 7 shows an alternative embodiment for how the lever 605 moves relative to the carriage brake 603. As illustrated in FIG. 7, the placement of the detents 604 relative to the pivot 701 and the levers 605 impacts if the cam surface is locked when the lever 605 is pushed away from the rail, similar to a car handbrake, or if is pushed toward the rail, similar to a bicycle handbrake.

In other embodiments, the latch may be an interference style as well, engaging a tooth profile along a toothed rack located on the back of the rail (not shown).

FIG. 8 is an example of a rotation lock. During a medical procedure, the user may want to perform tasks to manipulate the instrument (e.g., needle) that require the needle and an accessory (e.g. syringe) to rotate relative to each other. For example, after a needle insertion, the user may want to remove and/or install a syringe. A commonly used interface includes a screw type Luer lock.

To allow for single hand accessory manipulation, a rotational lock 801 may be applied to the needle. For example, as illustrated in FIG. 8, the rotational lock 801 may be a separate clip 802 that attaches to the carriage and adapter and holds the hub on the back of the needle. The rotation lock 801 may include ratcheting arms 803 to maintain position.

In other embodiments, the rotational lock 801 may be integrated into just the instrument hub, just the instrument adapter, or just the carriage and mechanically actuated (not shown). In the instrument hub approach, a flange may be created, where the collision into the carriage resists the rotational motion being applied to the instrument.

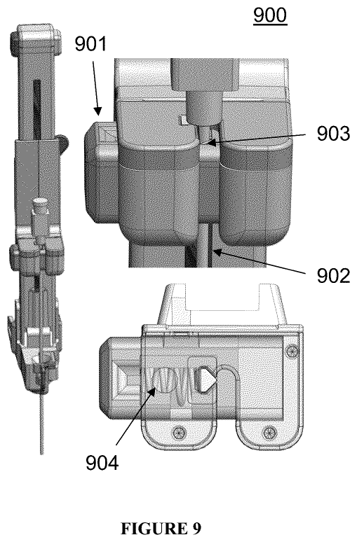

FIGS. 9-11 are examples of an instrument adapter. The instrument interface may be connected to the instrument by grabbing the needle hub or the needle shaft. Grabbing the needle hub may be advantageous because it does not consume any functional working length of the needle and may be combined with the rotation lock feature to reduce or prevent rotation of the instrument. Grabbing the needle shaft conversely may be advantageous because it allows a single component to interface with a wide range of needle-like instruments. The only variable to control is adjusting for different needle diameters. The types of mechanisms that meaningfully hold the needle shaft are not conducive to quick lateral attachment to the carriage. To overcome this, an instrument adapter is used between the instrument and the carriage.

One end of the instrument adapter is a universal needle gripping mechanism, and on the other is a quick attach/release mechanism to attach to the carriage. The needle gripping mechanism and be any mechanism that meaningfully grips the needle shaft and can vary diameters to accommodate typical instrument needle gauges (e.g. 10-27). A spring-loaded gripper 900, as illustrated in FIG. 9, may be held in one hand and released by pushing a button 901. The needle 902 is threaded through the opening 903 and the button 901 is released to apply grip force to the needle 902 by way of a spring 904. This gripping device 900 may be any gripping mechanism that includes set screws, clamps, ratchets, and the like.

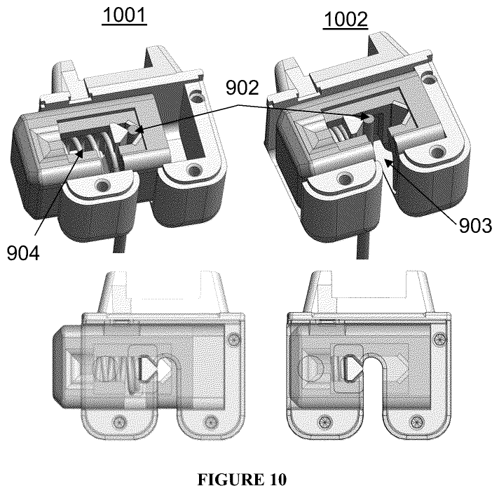

FIG. 10 illustrates the movement of the spring-loaded gripper 900 from both a top and planar view. The spring-loaded gripper 900 may move from a closed position 1001 to an open position 1002 when the button 901 is engaged.

Advantageously, the attachment of the instrument adapter to the needle may be accomplished using two hands and is a preparatory step to the procedure. Once installed, the instrument is ready to be quickly installed and removed from the carriage with one hand by way of the quick attach/release mechanism of the instrument adapter.

An alternate embodiment of this approach involves a cut out in the plunger to allow the instrument adapter to be removed from the instrument when the instrument shaft is already in the patient. This allows lateral attachment and release of the instrument from the adapter. However, quick attach/release is facilitated by the adapter carriage interface as described in further detail below.

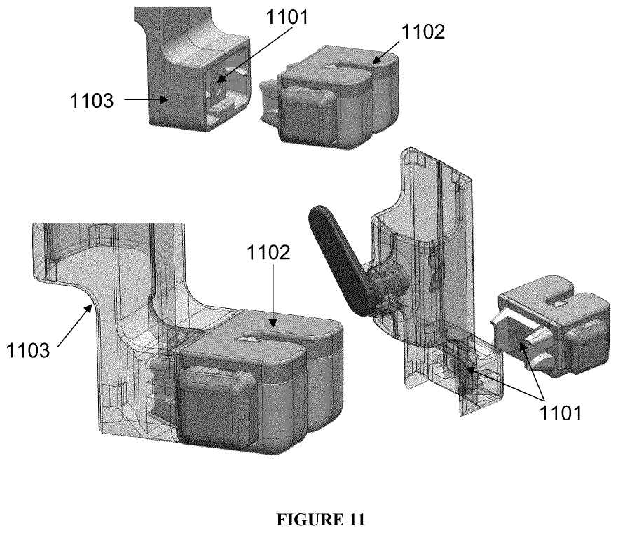

The quick attach/release mechanism of the instrument adapter provides one handed quick installation and removal of the needle to the carriage. As illustrated in FIG. 11, magnets 1101 may be used to hold the instrument adapter 1102 in place on the carriage 1103, and provide assisting forces during quick installation. The geometry of the interface surfaces can be designed to retain or allow the instrument to be attached/removed laterally as well as axially. Lateral attach/release can be beneficial for cases where the needle is being left in the patient while the robot is being removed (e.g. catheter installation). Axial attach/release can be beneficial for cases where the needle is being repeatedly inserted and removed (e.g. biopsies requiring fine needle aspiration). However, the instrument also needs some lateral and axial retention to prevent it from inadvertently detaching from the carriage during insertion as it is exposed to forces from the operator's hand and from the patient's tissue. The geometry can be customized to increase or decrease the amount of retention as necessary for the clinical application.

Lateral attach/release may be important for cases where the needle is being left in the patient while the robot is being removed (e.g. catheter installation). Axial attach/release may be important for cases where the needle is being repeatedly inserted and removed (e.g. biopsies requiring fine needle aspiration). The attachment mechanism need not be limited to magnets. Other approaches may include spring loaded or compliant clamps, slip fits, friction fits, bayonet clips, the like, or combinations of these approaches to vary the force needed to detach the instrument at different angles from the rail.

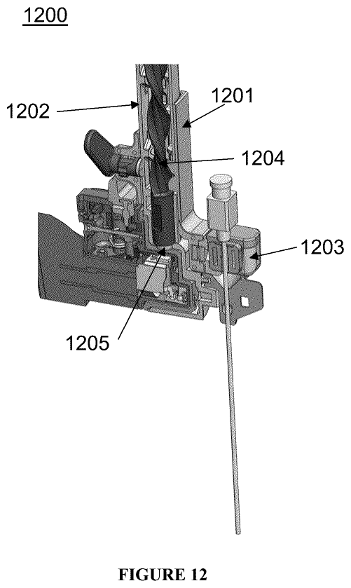

FIG. 12 is an example of the insertion rail The insertion rail 1200 may include two functional features. The first is structural support 1201 for the instrument, carriage, and top button. This structural support 1201 may allow the user to more intuitively adjust trajectories from the back end of the instrument using a stiff member rather than the flexible member of a thin needle shaft. The second feature is a mechanism to directly measure motion 1202 of the instrument by way of the carriage.

As illustrated in FIG. 12, the carriage 1203 interfaces with a helical screw 1204 that spins as the carriage 1204 moves up and down the insertion rail 1200. A magnet 1205 may be placed on the rotating shaft in proximity to the robot electronics. The electronics read the magnetic field orientation and compute an angle associated with the screw. Tracking the angle of the screw over time results in a direct measurement of the motion of the instrument along the rail. Because the communication of the screw motion to the electronics is transmitted via magnetic field, a sterile barrier may be maintained between the magnet 1205 and electronics (not shown). The magnet 1205 and screw 1204 are inexpensive components and may be disposed of per procedure, where the electronics remain in the capital components of the robot.

Alternative embodiments may include: (1) mechanically coupling the screw to a separate member that spins the magnet, (2) using cable drive or belts that run the length of the rail and is coupled to a rotary member, such as a capstan, that similarly uses a magnet interface to impart positional information to the robot electronics, or (3) making the rail capital and using a coil or magnet in the carriage that creates a current or magnetic field along sensors placed at regular intervals along the length of the rail.

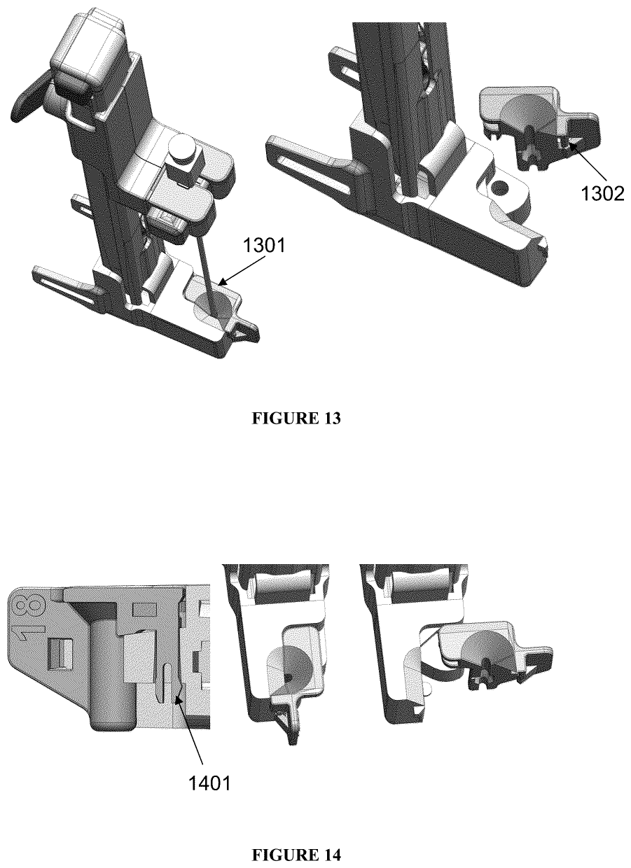

FIG. 13 is an example of a guide constraining needle motion. In an embodiment, the instrument may travel through a guide 1301 very close to the patient skin to provide accurate targeting. As illustrated in FIG. 13, an insert 1302 may be used to accommodate a precision fit between the instrument guide and the instrument (e.g., needle). Each instrument may use a gauge specific insert.

In other embodiments, the guide may be an adjustable mechanism that manipulates an aperture to accommodate different needle gauges.

FIG. 14 is an example of a guide lateral release In an embodiment, the guide 1401 may release the instrument (e.g., needle) laterally. Lateral release means that the guide may be removed from the needle with a motion that is orthogonal to the needle's longitudinal axis. This provides both a safety feature and one that enables clinical workflow. For example, if the robot malfunctions, the user may remove the robot from the needle and convert to a manual procedure. If the needle must remain inserted in the patient, the robot may be laterally removed by way of a guide lateral release. This may also be part of a clinical workflow where the robot is removed from a needle which remains in the patient.

The lateral release may be facilitated by the insert being hinged such that it can open the edge that is away from the robot. Other embodiments may include the insert popping off completely, or an adjustable insert that opens wide enough to expose a relief section of the guide to release the needle.

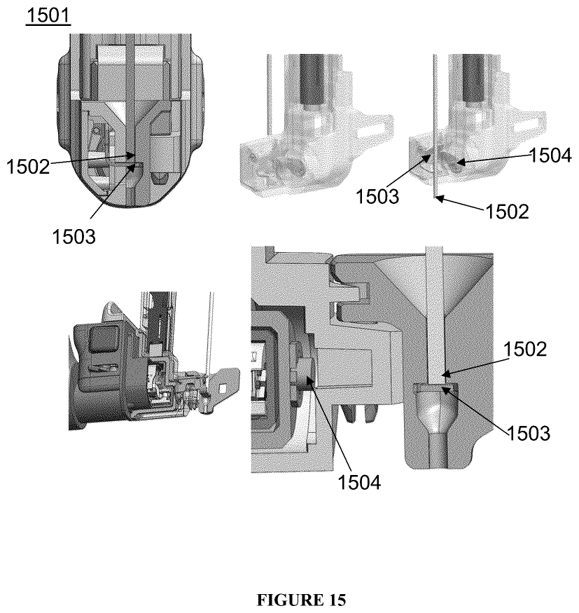

FIG. 15 is an example of a homing switch. The instrument insertion measurement described above provides incremental or relative instrument measurements. This is because the length of the needle may be unknown in some circumstances. The homing switch 1501 may detect correct instrument length to correctly display the location of the instrument tip on an image (e.g., ultrasound imaging) during the procedure. This may be accomplished by measuring the location of the instrument tip relative to the robot based on the homing switch signal and the insertion measurement from the carriage and helical screw. If the location of the instrument tip is known relative to the robot, then the tip location in the image may be computed using robot kinematics and image calibrations.

One embodiment, as illustrated in FIG. 15, may detect the instrument tip 1502, which may accommodate computing the tip location. The needle 1502 may trip a physical interference switch or door 1503. Motion of the door 1503 may be detected by electronics located directly behind the door 1503 in the capital components of the robot. When the door 1503 is actuated, a magnet 1504 moves and is detected across the sterile barrier. The switch or door 1503 location is known to the robot, and thus the location of the tip 1502 may be computed by fusing the encoder information from the insertion rail and the state of the switch 1504. Further, the switch state 1504 may be used to determine if the needle is being inserted, or if there is no needle insertion.

Similar to the paradigm described for the insertion screw magnet, the motion of the switch may be communicated across the sterile barrier by detecting motion of a magnetic field. Other embodiments may include inductive sensors, or directly pushing a button across a compliant section of the sterile barrier.

In another embodiment, the user may explicitly align the tip of the needle and manually communicate the homing step. In such a solution, the homing “switch” 1501 is a user interaction like clicking a button on a touch screen or on the robot to denote that the needle tip is at a known location or reference point.

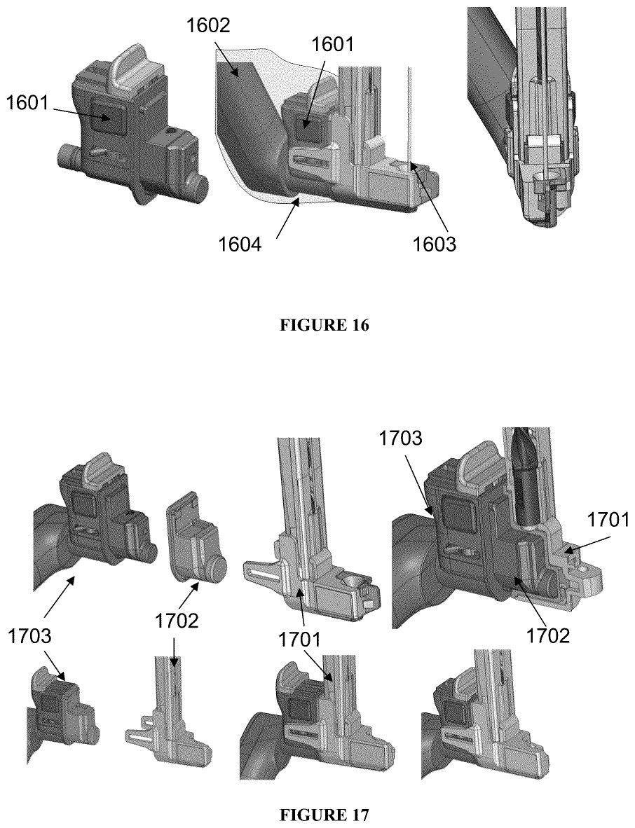

FIG. 16 is an example of the lower button. The lower button 1601 may be used to deploy the arm 1602 manually and perform arm adjustment to align the tip of the needle guide 1603 with the desired instrument insertion site on the skin, which may be advantageous when performing procedures. As illustrated in FIG. 16, the lower button 1601 itself may be located on the capital component as part of the instrument interfaces electronics. However, care must be taken to cover the lower button 1601 to create a sterile barrier, while maintaining a tactile user interaction. As shown here, the instrument interface has sterile covers 1604 that protrude over the lower button 1601 interface, providing a nice tactile interaction and maintaining a sterile field. Similarly, the drape may simply cover the lower button 1601, allowing the user to push the lower button 1601 through the compliant drape. As shown, there may be lower buttons 1601 on both sides of the device, to maintain symmetry when switching between right- and left-hand configurations.

FIG. 17 is an example of a sterile adapter. The instrument interface 1701 may latch to the robot 1703 over a sterile adapter 1702. The sterile adapter 1702 may be a simple component that may be part of the drape assembly. It provides a rigid and accurate interface between the robot 1703 and the instrument interface 1701.

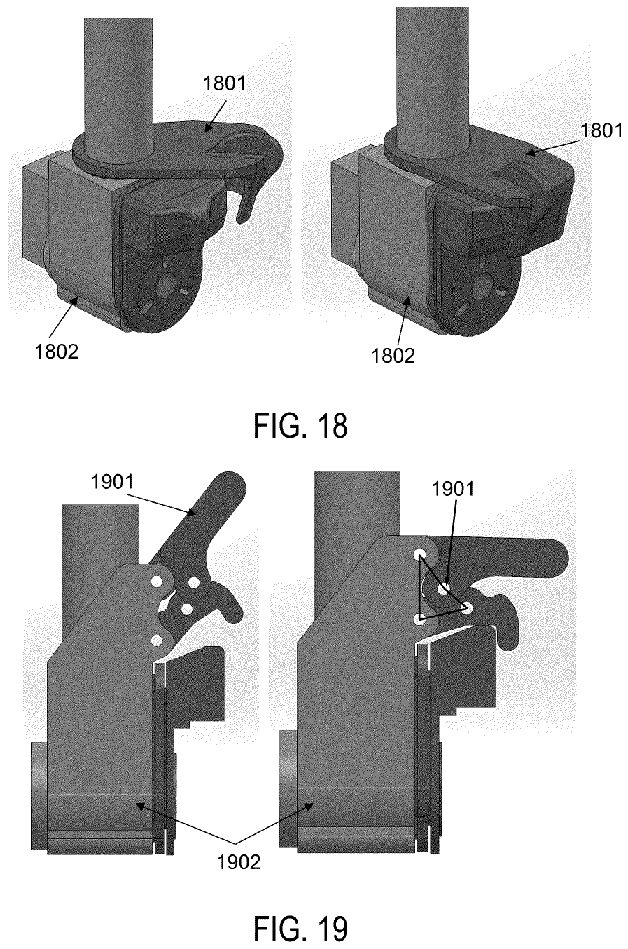

FIGS. 18-21 are examples of an instrument interface latch. The instrument interface latch may latch to the distal link (L3) of the robot and sandwich the sterile adapter in between.

As illustrated in FIG. 18, one embodiment shows where a latch component 1801 rotates and captures the robot 1802. As illustrated in FIG. 19, another embodiment involves an over-center mechanism 1901 to also grab and latch to the robot 1902.

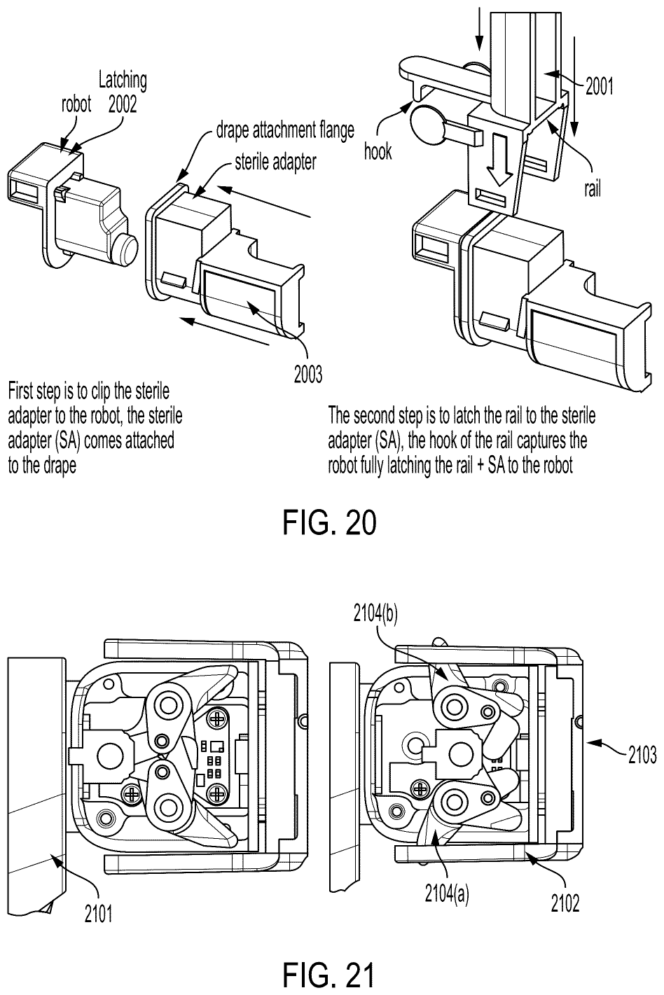

As illustrated in FIG. 20, in yet another embodiment, the instrument interface 2001 may be installed on the robot 2002 by latching over a section of the robot end effector containing complementary sensor electronics (e.g. encoders, buttons, hall sensors, etc.). The “sterile adapter” 2003 also houses the lower features of the instrument interface (e.g. instrument guide and homing switch). The rail may then slide onto the sterile adapter to complete the instrument interface assembly.

FIG. 21 is an example of a latching mechanism that is part of the capital components of the robotic end effector. In this embodiment, the moving components of the latch 2102 are positioned at the end effector 2101 and covered by the drape. A mechanism deploys the latching components 2102 to grab features of the rail to secure it to the robot 2103. FIG. 21 illustrates two lateral pawls 2104(a) and 2104(b) that rotate to grab the rail. The pawls 2104(a) and 2104(b) move into slot 2105 that allow the rail to be pulled onto the robot 2103. Further, the pawls 2104(a) and 2104(b) may be spring loaded to apply a preload to ensure that the rail is fully seated on the robot or sterile adapter and ensure a deterministic an accurate registration between the rail and the robot.

In this embodiment, the pawls 2104(a) and 2104(b) may be actuated by a sliding mechanism. The sliding mechanism may be connected to the rotating pawls 2104(a) and 2104(b) by an over centered linkage. This transmits linear motion to the two rotating pawls 2104(a) and 2104(b) simultaneously. Further, when the slider is moved past the over center mechanism it locks the pawls 2104(a) and 2104(b). Compliance between the pawls 2104(a) and 2104(b) and the linkage allow for the rail preload, as well as facilitates the mechanism moving through the over center position to lock the rail. This arrangement is advantageous since it allows the rail side mechanism to be a passive and simple pawl interface feature, which simplifies the rail and drives down cost. Cost is important since the rail is disposable. Conversely, the pawls 2014(a) and 2104(b) and latching mechanism may be more complex due to relaxed cost constraints more space on the capital robot side. The actuation mechanism shown is a slider, however it may also be a rotating switch, lever or the like.

An alternative embodiment may include a version where the sterile adapter is a simple interface between the robot and the instrument interface in its entirety. In this case, the entire instrument interface is one functional assembly. The sterile adapter may comprise a simple plate with a bonded drape that attaches to the robot on one side and the instrument interface on the other side.

In yet another embodiment, the drape and instrument interface may comprise one integrated unit where the drape goes over the robot and the instrument interfaces latches onto the robot without a sterile adapter.

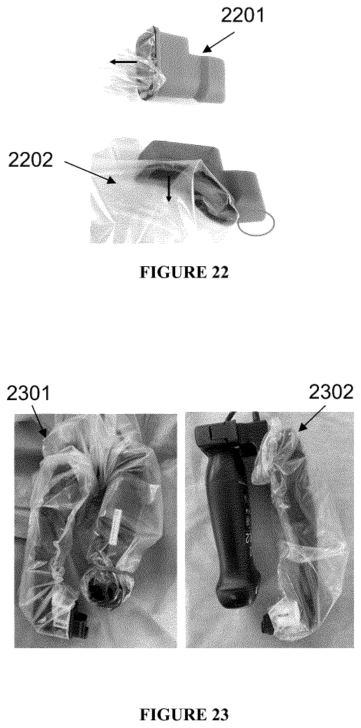

In yet another embodiment, the drape may simply cover the distal link of the robot and the instrument interface may attach over the compliant drape material. FIG. 22 is an example of a drape boned to an adaptor. A drape 2201 may be bonded to the back of the sterile adapter 2202 to provide a sterile barrier between the robot and procedure.

FIG. 22 illustrates how the drape may be bonded to two different sterile adapter 2201 geometries. The top image shows the drape 2202 in the latching figure. The bottom image shows an equivalent system architecture with a different drape bond geometry. The arrow points in the direction that the sterile adapter attaches to the robot end effector.

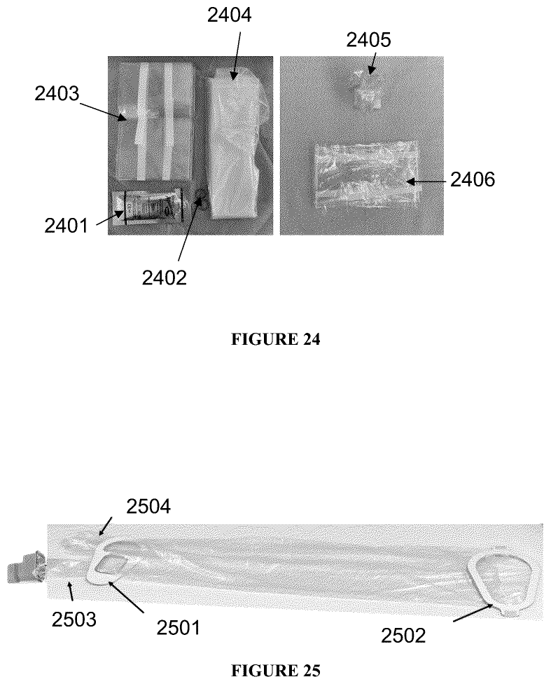

FIGS. 23-24 are examples of drape packages.

As illustrated in FIG. 23, there are two fully draped robots using a sterile adapter. On the left is the fully draped robot 2301 that creates a sterile barrier between the ultrasound probe, the entire robot, the lower part of the cable and the surgical site. On the right is an arm only drape that covers just the robot arm 2302.

Illustrated in FIG. 24 are examples of drape packages for a full drape and arm-only drape, including contents for each package. These embodiments show how consumables—including the folded drape, sterile adapter, inner and outer frame as well as other sterility accessories—may be provided to a user for clinical installation.

The full drape package may provide for completely draping the entire robot, transducer, and cables. The full drape package may include a full drape with sterile adapter (not shown), ultrasound gel packs 2401, two elastic bands, 2403, a surgical drape 2403, and packaging 2404. This drape may include a pocket for the imaging device (e.g., ultrasound probe), and a pocket for the robotic arm. The sterile adapter attaches to the pocket that is for the robot.

The arm only drape package may include the sterile adapter 2405 which is coupled with a shortened drape 2406 to provide a localized sterile field at the instrument. A hook or an elastic at the opposite end of the drape may be used to keep the arm drape extended over the arm of the robot. The goal of this drape is to maintain sterility around the instrument shaft while facilitating the semi-sterile conditions currently used, where the hand that operates the needle or instrument is considered sterile, while the other hand that holds the probe is considered nonsterile.

FIGS. 25-28 are examples of workflow for rapid installation of a full drape package. As illustrated in FIG. 25, an inner frame 2501 and outer frame 2502 are used during installation. The drape may further include a sterile adapter interface 2503 and a probe head pocket 2504.

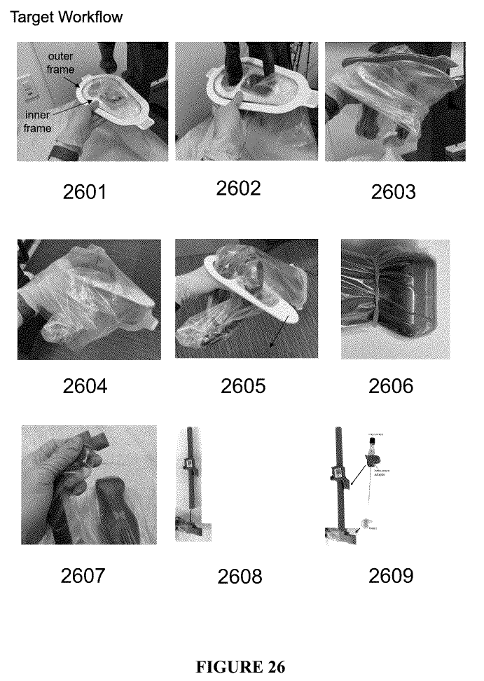

FIG. 26 is an example of a step by step workflow. First ultrasound gel may be applied to the inside of a drape pocket for the probe and lift frames 2601. The probe and arm may then align with the appropriate holes 2602. The system may then lift up from the stand 2603. The outer frame may then extend over the cable length 2604. Then the inner frame may be removed and discarded 2605. A rubber band may then attach around the probe head 2606. The sterile adapter is then aligned and latched onto the robot 2607. The rail part of the instrument interface may then attach to the sterile adapter 2608. Finally, the instrument adapter is installed and inserted 2609.

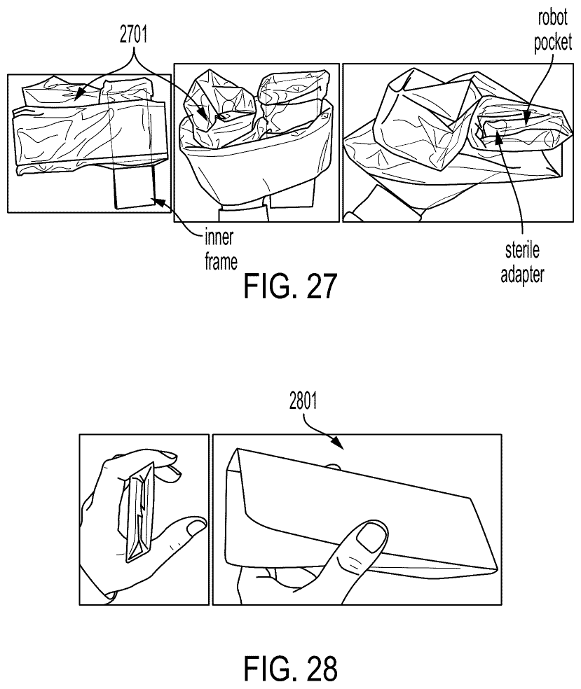

FIGS. 27 and 28 are examples of alternative embodiments to install the full drape package. One challenge with draping any attachments to an imaging device (e.g., ultrasound probe) is the management of the ultrasound gel inside of the probe pocket 2701, while maintaining sterility and quickly covering the attachment at the same time. This alternative embodiment may allow the user to use the same conventional approach of applying gel to the pocket 2701, and grabbing the pocket 2701 from the other side to keep the gel pressed up against the probe.

To attach the other pocket a small frame may be used. This frame comprises two key features: (1) the extension of the white frame outside of the rest of the drape allows the user to steer without their hand being restrained by the rest of the drape—which helps with control as well as accessibility across hand sizes, and (2) the card 2801 encloses the inner pocket but can be popped open, similar to moving boxes. This keeps the user from grabbing onto the wrong fold and helps keep the lumen open. The card 2801 is kept open to flex around the robot arm. The card is illustrated in FIG. 28.

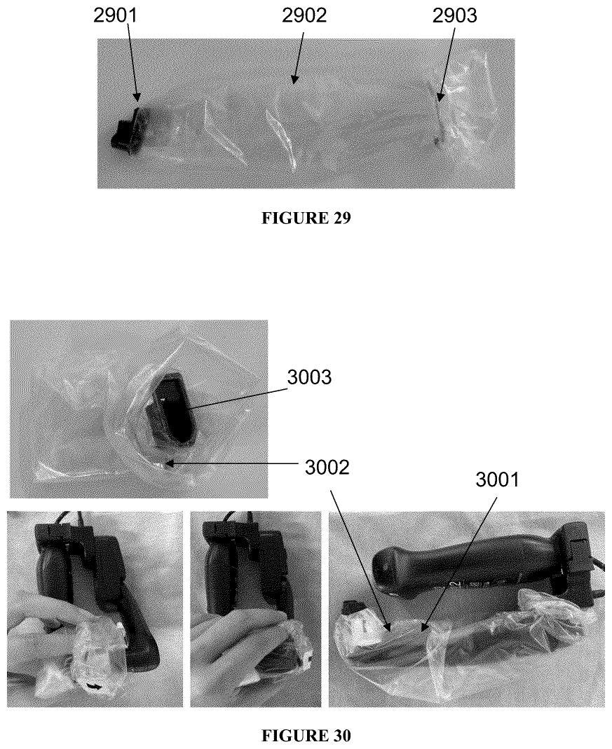

FIGS. 29 and 30 are examples of the arm only drape. As illustrated in FIG. 29, the sterile adapter 2901 is coupled with a shortened drape 2902 to provide a localized sterile field at the instrument. A hook or an elastic 2903 at the opposite end of the drape 2902 can be used to keep the arm drape extended over the arm of the robot. The goal of this drape is to maintain sterility around the instrument shaft while facilitating the semi-sterile conditions currently used, where the hand that operates the needle or instrument is considered sterile, while the other hand that holds the probe is considered nonsterile.

FIG. 30 illustrates the workflow to drape 3002 the instrument 3001, and install the instrument interface at the sterile adapter 303 to create a sterile field and prep the system for procedure readiness.

The foregoing description of the preferred embodiments of the present invention is by way of example only, and other variations and modifications of the above-described embodiments and methods are possible in light of the foregoing teaching. The various embodiments set forth herein may be implemented utilizing hardware, software, or any desired combination thereof. For that matter, any type of logic may be utilized which is capable of implementing the various functionality set forth herein. Components may be implemented using a programmed general-purpose digital computer, using application specific integrated circuits, or using a network of interconnected conventional components and circuits. Connections may be wired, wireless, modem, etc. The embodiments described herein are not intended to be exhaustive or limiting.

What is claimed:

1. A sterile disposable interface for percutaneous instruments, the sterile disposable interface comprising:
an insertion rail;
a carriage, wherein the carriage is attached to the insertion rail; and
a carriage position sensor, wherein the carriage position sensor determines a position of the carriage relative to a position of the insertion rail as the carriage moves along the insertion rail;
wherein the carriage position sensor communicates the position of the carriage across a sterile barrier.

2. The sterile disposable interface of claim 1, further comprising an instrument adapter.

3. The sterile disposable interface of claim 1, further comprising an instrument guide.

4. The sterile disposable interface of claim 1, further comprising a homing switch.

5. The sterile disposable interface of claim 1, wherein the sterile disposable interface laterally disengages from the percutaneous instrument at the carriage.

6. The sterile disposable interface of claim 1, further comprising a top lock.

7. The sterile disposable interface of claim 1, further comprising a carriage brake.

8. The sterile disposable interface of claim 1, further comprising a rotation lock.

9. The sterile disposable interface of claim 1, further comprising an adjustment button.

10. The sterile disposable interface of claim 1, further comprising a sterile drape.

11. The sterile disposable interface of claim 1, wherein the carriage position sensor determines the position of the carriage by rotating a magnetic field.
